(19) United States
(12) Patent Application Publication
(10) Pub. No.: US 2026/0325433 A1
(43) Pub. Date: Oct. 22, 2026

(54) PERSONAL CLEANSING COMPOSITION

(71) Applicant: Conopco, Inc., d/b/a UNILEVER, Englewood Cliffs, NJ (US)

(72) Inventors: Andrew Richard AVERY, Ellesmere Port, Cheshire (GB); Hailey KELSO, Chester (GB); Rongrong ZHOU, Wirral, Merseyside (GB)

(21) Appl. No.: 19/486,317

(22) PCT Filed: Jun. 11, 2024
(86) PCT No.: PCT/EP2024/066047

§ 371 Date: Nov. 20, 2025

(30) Foreign Application Priority Data
Jun. 29, 2023 (EP) 23182472.3

(51) Int. Cl. A61K 8/92; A61K 8/06; A61K 8/37; A61K 8/44; A61K 8/46; A61K 8/73; A61Q 5/02; A61Q 5/12

(52) U.S. Cl. CPC A61K 8/922; A61K 8/068; A61K 8/375; A61K 8/442; A61K 8/463; A61K 8/73; A61Q 5/02; A61Q 5/12; A61K 2800/34; A61K 2800/5426; A61K 2800/596; A61K 2800/805

(57) ABSTRACT

A silicone free aqueous cleansing composition, comprising: i) from 3 to 15 wt % of a sulphated anionic surfactant; ii) from 0.5 to 5 wt % of a zwitterionic surfactant; iii) from 0.1 to 1 wt % a triglyceride oil comprising at least 30% of fatty chains having a carbon-carbon chain length of 12; iv) from 0.01 to 0.1 wt % caprylic acid; and v) from 0.1 to 2 wt % of a cationic polysaccharide deposition polymer; wherein the ratio of triglyceride oil to caprylic acid is 5:1 to 15:1; wherein the composition is in the form of a microemulsion; and wherein the composition is free from silicone, provides frizz reduction and conditioning benefits to hair.

### FIELD OF THE INVENTION

The invention is in the field of personal care cleansing products that provide conditioning benefits and are free from silicone.

### BACKGROUND OF THE INVENTION

Consumers desire hair treatments, such as shampoos, that provide conditioning benefits, for example smoothness, ease of comb and softness to their hair.

Conditioning benefits are desired and perceived by the consumer both during and after the wash process. Thus, conditioning is evaluated both when the hair is wet and when it is dry. As such, it is desirable that rinse-off haircare products such as shampoos provide multiple benefits at different stages of use. Wet conditioning and dry conditioning are different benefits and are typically delivered in different ways.

Silicones are primarily used to provide conditioning benefits at the post drying stages. In silicone-free formulations alternative approaches are required to provide such benefits.

Oils have been used as an alternative to silicone in hair products that comprise sulphate free anionic surfactants. However, incorporation of the oil into a base comprising sulphated anionic surfactants presents stability problems, with the oil separating over time, as well as rheological problems, the oil causing viscosity reduction. Sulphated surfactants allow ease of formulation and provision of good foaming properties and viscosity in cleansing compositions.

WO 21/175499 (Unilever) discloses personal cleansing composition for the mitigation of dandruff comprising: i) a cleansing surfactant comprising an amphoteric surfactant and an anionic surfactant which if ethoxylated has a degree of ethoxylation of less than 3, in which the ratio of anionic surfactant to amphoteric surfactant is less than 5:1; ii) an oil phase comprising at least one triglyceride oil; iii) an aliphatic fatty acid or salt thereof having a carbon chain length from C4 to C10 and iv) a piroctone compound; in which the composition has a pH at 20 °C of 6 or less and the weight ratio of aliphatic fatty acid to triglyceride oil is from 1:2 to 5:1.

Despite the prior art there remains a need for improved conditioning in the absence of silicone in compositions that comprise sulphated surfactants.

We have now found that surprisingly, frizz reduction as well as wet and dry lubrication can be delivered from silicone free cleansing compositions having a sulphated anionic surfactant, that comprise a triglyceride oil and caprylic acid in combination with a cationic deposition polymer. The compositions are in the form of a microemulsion and are stable and have excellent viscosity and foaming properties.

### SUMMARY OF THE INVENTION

In a first aspect there is provided an aqueous cleansing composition, comprising:
i) from 3 to 15 wt % of a sulphated anionic surfactant;
ii) from 0.5 to 5 wt % of a zwitterionic co-surfactant;
iii) from 0.1 to 1 wt % of a triglyceride oil comprising at least 30% of fatty chains having a carbon-carbon chain length of 12;
iv) from 0.01 to 0.1 wt % caprylic acid; and
v) from 0.1 to 2 wt % of a cationic polysaccharide deposition polymer;
wherein the ratio of oil to caprylic acid is 5:1 to 15:1;
wherein the composition is in the form of a microemulsion; and
wherein the composition is free from silicone.

A second aspect provides a method of treating hair with a composition of the first aspect, comprising the step of applying the composition to the hair.

A third aspect provides a use of a composition of the first aspect to condition hair. Preferably the conditioning is provided by a reduction in friction on dry or wet hair, preferably wet hair.

Use of a composition of the first aspect to reduce frizz of hair is also provided, compared to a similar composition that does not comprise caprylic acid and triglyceride oil.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the final personal care composition, unless otherwise specified. It should be noted that in specifying any ranges of values, any particular upper value can be associated with any particular lower value.

### The Sulphated Anionic Surfactant

The composition of the invention includes a sulphated anionic surfactant, preferably selected from sodium laureth ether sulphate (SLES), sodium pareth ether sulphate (SPES) and mixtures thereof. Preferably the anionic surfactant has an average degree of ethoxylation of from 1EO to 3EO, more preferably 1EO to 2EO.

In a typical composition of the invention the level of anionic surfactant will generally range from 3 to 15 wt %, preferably from 5 to 14 wt %, most preferably from 8 to 13 wt % (by weight based on the total weight of the composition and 100% activity).

### The Zwitterionic Co-Surfactant

The composition of the invention includes a zwitterionic co-surfactant, which is preferably selected from a betaine, cocamide monoethanolamide (CMEA) and mixtures thereof.

The level of zwitterionic co-surfactant is from 0.5 to 5 wt %, preferably from 1 to 4 wt %, more preferably from 1.5 to 3 wt % of a zwitterionic surfactant, by weight based on the total weight of the composition and based on 100% active level.

Betaines that are suitable for use in the present invention can be represented by the general formula:

R¹⁰[C(O)NH—(CH₂)_{y}]_{z}−N⁺(R¹¹)(R¹²)CH₂CO₂⁻ (IV)

where R¹⁰ is C6 to C30, more particularly C6 to C24 alkyl, z is 0 or 1, R¹¹ and R¹² are independently alkyl, hydroxyalkyl or carboxyalkyl of 1 to 3 carbon atoms, and y is 2 or 3; and salts thereof. In one embodiment, at least half of the groups R¹⁰ are C8 to C18 alkyl. In another embodiment, at least half of the groups R¹⁰ are C 10 to C14 alkyl. R¹⁰ may be saturated or unsaturated. In one embodiment, R¹⁰ is derived from coconut oil or palm kernel oil. In one embodiment R¹¹ and R¹² are methyl.

The formula (IV) betaines include the simple betaines:

R¹⁰—N⁺(R¹¹)(R¹²)CH₂CO₂⁻ (IVa)

where R¹⁰, R¹¹, and R¹² are as described above, and the amidobetaines:

R¹⁰C(O)NH—(CH₂)_{y}—N⁺(R¹¹)(R¹²)CH₂CO₃⁻ (IVb)

where R¹⁰, R¹¹, R¹², and y are as described above.

Preferred betaines are oleyl betaine, caprylamidopropyl betaine, lauramidopropyl betaine, isostearylamidopropyl betaine, and cocoamidopropyl betaine and mixtures thereof. Most preferably, the zwitterionic co-surfactant is cocamidopropyl betaine.

Most preferably, the cosurfactant is selected from cocamidopropylbetaine (CAPB), cocamide monoethanolamide (CMEA) and mixtures thereof.

The weight ratio of the anionic surfactant (i) to the zwitterionic co-surfactant (ii) is preferably from 3:1 to 10:1, more preferably from 3.5:1 to 10:1, even more preferably from 5:1 to 10:1, most preferably from 6:1 to 9:1.

### The Triglyceride Oil

The composition of the invention includes an oil, which is a triglyceride oil comprising at least 30%, preferably from 30 to 100%, more preferably from 40 to 75 wt %, most preferably from 40 to 60% of the fatty chains have a carbon-carbon chain length of 12.

The triglyceride oil is preferably selected from coconut oil, palm kernel oil, algal oil and mixtures thereof, most preferably coconut oil.

The oil is present in an amount of from 0.1 to 1 wt %, preferably from 0.15 to 0.5 wt %, more preferably from 0.2 to 0.4 wt %, by weight based on the total weight of the composition.

### The Caprylic Acid

The composition of the invention comprises caprylic acid.

The caprylic acid is present in an amount of from 0.01 to 0.1 wt %, preferably from 0.025 to 0.9 wt %.

The weight ratio of tryglyceride oil to caprylic acid is 5:1 to 15:1, preferably from 6:1 to 12:1.

### The Cationic Polysaccharide Deposition Polymer

The composition of the invention includes a cationic polysaccharide deposition polymer.

The cationic polysaccharide deposition polymer is present in an amount of from 0.1 to 2 wt %, preferably from 0.15 to 1.5 wt %, most preferably from 0.2 to 1.0 wt % based on the total weight of the composition.

The cationic polysaccharide deposition polymer is preferably selected from cationic dextrans, cationic cellulose derivatives and cationic polygalactomannans.

A preferred cationic deposition polymer is selected from cationic polygalactomannans having a mean charge density at pH7 from 0.2 to 2 meq per gram. Such polymers may serve to enhance the delivery of conditioning agents from the composition to the skin and/or hair surface during consumer use, thereby improving the conditioning benefits obtained. Mixtures of cationic deposition polymers may be employed.

The term “charge density” in the context of this invention refers to the ratio of the number of positive charges on a monomeric unit of which a polymer is comprised to the molecular weight of the monomeric unit. The charge density multiplied by the polymer molecular weight determines the number of positively charged sites on a given polymer chain.

The polygalactomannans are polysaccharides composed principally of galactose and mannose units and are usually found in the endosperm of leguminous seeds, such as guar, locust bean, honey locust, flame tree, and the like. Guar flour is composed mostly of a galactomannan which is essentially a straight chain mannan with single membered galactose branches. The mannose units are linked in a 1-4-3-glycosidic linkage and the galactose branching takes place by means of a 1-6 linkage on alternate mannose units. The ratio of galactose to mannose in the guar polymer is therefore one to two.

Suitable cationic polygalactomannans for use in the invention include polygalactomannans, such as guars, and polygalactomannan derivatives, such as hydroxyalkyl guars (for example hydroxyethyl guars or hydroxypropyl guars), that have been cationically modified by chemical reaction with one or more derivatizing agents.

Derivatizing agents typically contain a reactive functional group, such as an epoxy group, a halide group, an ester group, an anhydride group or an ethylenically unsaturated group, and at least one cationic group such as a cationic nitrogen group, more typically a quaternary ammonium group. The derivatization reaction typically introduces lateral cationic groups on the polygalactomannan backbone, generally linked via ether bonds in which the oxygen atom corresponds to hydroxyl groups on the polygalactomannan backbone which have reacted.

Preferred cationic polygalactomannans for use in the invention include guar hydroxypropyltrimethylammonium chlorides.

Guar hydroxypropyltrimethylammonium chlorides for use in the invention are generally comprised of a nonionic guar gum backbone that is functionalized with ether-linked 2-hydroxypropyltrimethylammonium chloride groups, and are typically prepared by the reaction of guar gum with N-(3-chloro-2-hydroxypropyl)trimethylammonium chloride.

Cationic polygalactomannans for use in the invention (preferably guar hydroxypropyltrimethylammonium chlorides) generally have an average molecular weight (weight average molecular mass (Mw) determined by size exclusion chromatography) in the range 500,000 to 3 million g/mol, more preferably 800,000 to 2.5 million g/mol.

Cationic polygalactomannans for use in the invention generally have a charge density ranging from 0.5 to 1.8 meq/g.

Preferably the cationic polygalactomannans are selected from guar hydroxypropyltrimethylammonium chlorides having a charge density ranging from 0.5 to 1.8 meq/g (and mixtures thereof).

The cationic charge density of the polymer is suitably determined via the Kjeldahl method as described in the US Pharmacopoeia under chemical tests for nitrogen determination.

Specific examples of preferred cationic polygalactomannans are guar hydroxypropyltrimonium chlorides having a cationic charge density from 0.5 to 1.1 meq/g.

Also suitable are mixtures of cationic polygalactomannans in which one has a cationic charge density from 0.5 to 1.1 meq/g, and one has a cationic charge density from 1.1 to 1.8 meq per gram.

Specific examples of preferred mixtures of cationic polygalactomannans are mixtures of guar hydroxypropyltrimonium chlorides in which one has a cationic charge density from 0.5 to 1.1 meq/g, and one has a cationic charge density from 1.1 to 1.8 meq per gram.

Cationic polygalactomannans for use in the invention are commercially available from Solvay as JAGUAR® C13S, JAGUAR® C14 and JAGUAR® C17. Also Esaflor OX 14B available from Lamberti.

In a preferred composition according to the invention the cationic polygalactomannans are selected from guar hydroxypropyltrimethylammonium chlorides having a charge density ranging from 0.5 to 1.8 meq/g (and mixtures thereof), at a level ranging from 0.15 to 0.2% by weight based on the total weight of the composition.

Examples of preferred cationic cellulose derivatives for use in the invention include poly(1,2-oxyethanediyl)-2-hydroxy-3-trimethylammonium propyl chloride cellulose ethers (INCI: Polyquaternium-10).

Suitable cationic dextran polymers are described in WO2022/240665A1 at paragraph [0005].

In the context of the invention, by silicone free is meant having less than 0.15 weight %, more preferably less than 0.1 weight %, even more preferably less than 0.05 weight %, still more preferably less than 0.001 weight %, yet preferably less than 0.0001 weight %, and most preferably 0 weight % of silicone by weight of the total composition.

The compositions of the invention are preferably free from anti-dandruff agents, for example piroctone olamine.

The microemulsion contains worm-like micelles. The compositions are stable and have excellent viscosity. The composition can be transparent.

The aqueous treatment composition will generally comprise at least 60%, preferably at least 70% and more preferably at least 80% water (by weight based on the total weight of the composition). Preferably, the composition comprises no more than 99% and more preferably no more than 98% water (by weight based on the total weight of the composition).

The compositions may be made by mixing all ingredients, including the cationic deposition polymer, to make a shampoo base, then adding the triglyceride oil and caprylic acid. In an optional step, the pH may be adjusted with citric acid and the viscosity adjusted with sodium chloride or Polypropylene glycol P400.

Embodiments of the invention will now be illustrated by the following examples.

### EXAMPLES

### Example 1: Shampoo Compositions 1 in Accordance with the Invention and Comparative Composition A and B

Three hair shampoo formulations were prepared and used to treat hair prior to hair assessments. The compositions are given in Table 1.

TABLE 1
Ingredients (wt %) of Compositions 1 in accordance with
the invention and comparative composition A and B.

| | Amount (wt %) | | |
|---|---|---|---|
| Ingredient | 1 | A | B |
| Guar Hydroxypropyltrimonium | 0.25 | 0.25 | 0 |
| Chloride | | | |
| Sodium Laureth Ether Sulfate | 12.0 | 12.0 | 12.0 |
| Co-surfactant | 1.6 | 1.6 | 1.6 |
| Caprylic acid | 0.025 | 0 | 0.025 |
| Coconut Oil | 0.225 | 0 | 0.225 |
| Citric acid to required pH | 4.5 | 4.5 | 4.5 |
| Water & minors | to 100 | to 100 | to 100 |
| (including perfume, preservative, | | | |
| and thickener / thinner) | | | |

Formulations were made as follows: All ingredients were mixed to make a shampoo base, then coconut oil and caprylic acid were added in compositions in accordance with the invention. In an optional step, the pH was adjusted with citric acid and the viscosity adjusted with sodium chloride or Polypropylene glycol P400.

### Example 2: Treatment of Hair with Compositions 1, A and B and Hair Expansion Volume Measurements

The hair used was dark brown European curly hair, in switches of 2 g weight and 10 inch length.

The hair was treated with Compositions 1, A and B as follows:—

Hair was first treated with a cleansing shampoo using the following method:—

The hair fibres were held under running water for 30 seconds, shampoo applied at a dose of 0.1 ml of shampoo per 1 g of hair and rubbed into the hair for 30 seconds. Excess lather was removed by holding under running water for 30 seconds and the shampoo stage repeated. The hair was rinsed under running water for 30 seconds.

The wet hair was then treated with Shampoos 1, A or B using the following method:—Shampoo was applied to the wet hair at a dose of 0.1 g of shampoo per 1 g of hair and rubbed into the hair for 30 seconds. Excess lather was removed by holding under running water for 30 seconds and the shampoo stage repeated. The hair was rinsed under running water for 30 seconds, and excess water removed.

4 replicates hair switches were prepared for each shampoo. The hair switches were photographed in a controlled humidity chamber after dried in humidity chamber at 20 °C. 50% RH overnight; then hair switches were combed and photographed again. Hair expansion volumes were analyzed using image analysis software.

Both before comb and after comb hair expansion volume measured on hair switches treated with Shampoos 1, A and B at 20 °C 50% RH are shown in Table 2.

TABLE 2
Mean hair expansion volume (mm²) for hair treated
with Shampoos 1, A and B at 20 °C, 50% RH

| | 1 | A | B |
|---|---|---|---|
| Before comb | 7526 | 9325 | 12939 |
| After comb | 11077 | 13749 | 15614 |

Irrespective of whether hair is combed or not, Shampoo 1 produces lower hair expansion volume than comparative shampoos A and B.

### Example 3: Treatment and Volume Measurement of Untreated Switches for Calculation of Frizz Reduction

4 hair switches (2g×10″) were washed with a cleansing shampoo. The switches were dried in a high temperature environment for 60 to 80 minutes (50 °C). Once the switches were fully dry, they were combed several times to create an extremely frizzy switch. Frizz images were taken using the Image Analysis Volume Rig. The images were taken for each of hair switches to obtain a 3D volume area.

Before comb hair expansion volume of each treated hair switch was compared with untreated (extremely frizzy) switch volume values. Frizz reduction can thus be calculated.

% Frizz reduction=((¹base wash volume−²treated volume)/base wash volume)*100.

¹ volume of switches after cleaning with shampoo and frizzed up with a comb providing maximum frizz² volume of treated switches after drying at 20 °C. 50% RH overnight

Frizz reduction (%) was for each switch. The average of 4 switches is given in Table 3.

TABLE 3
Frizz Reduction for Hair Treated with Shampoos 1, A and B

| Shampoo | Average % Frizz Reduction |
|---|---|
| 1 | 54.7 |
| A | 43.9 |
| B | 22.1 |

The above table shows that Shampoo 1 has better frizz reduction than Shampoo A and B.

### Example 4: Salon Test with Compositions 1 and A

Half-head expert salon test was conducted, in which the performance of Shampoo 1 and Shampoo A was compared over a standard set of attributes at a number of key stages. Thirty-six panelists were recruited, that were aged between 16-65 years old, had straight to wavy and medium to coarse hair. The assessor applied the products in a standard half-head method, they recorded the side delivering the higher performance to the attribute, and the products were randomized. The assessment was forced choice, either left or right side and the data was collected via a tablet on Compusense. The attributes of significant difference are listed in Table 4.

TABLE 4
Significantly different attributes from half-
head salon study, where half head was washed
with Shampoos 1 and the other half with Shampoo A

| | | |
|---|---|---|
| Expert Evaluation | 1 | A |

| | | | |
|---|---|---|---|
| Shampoo | Ease of Rinse | x | |
| Rinsing | | | |
| Shampoo | Slippery feel under running water | x | |
| Rinsing | | | |
| Damp Stage | Ease of damp detangling | x | |
| Damp Stage | Slippery feel when damp | x | |
| Dry Stage | Ease of Styling | x | |
| Dry Stage | Alignment | x | |
| Dry Stage | Most Fluffy / Frizzy look | | x |
| Dry Stage | Ease of dry combing | x | |
| Dry Stage | Smooth feel | x | |
| Dry Stage | Softness | x | |
| Dry Stage | Dryness | | x |
| Dry Stage | Dryness of Ends | | x |
| Dry Stage | Level of Hold | x | |

“x” indicates highest level of attribute

Comparing with Shampoo A, Shampoo 1 delivers better wet/dry lubrication and provides conditioning and styling benefits. In shampoo rinsing stage, shampoo 1 delivers better slippery feel under running water and ease of rinse; in damp stage, Shampoo 1 delivers better ease of damp detangling and slippery feel when damp; in dry stage, Shampoo 1 delivers better alignment, ease of styling, ease of dry combing, smooth feel, softness, and level of hold while less frizz and less dryness.

### Example 4: Compositions D0 and F0 in Accordance with the Prior Art

Two comparative examples, D0 and F0, were prepared corresponding to Compositions D and F of WO21175499.

TABLE 5
Ingredients (wt %) of Comparative Compositions D0 and
F0, corresponding to Compositions D and F of WO21175499

| Ingredient | D0 | F0 |
|---|---|---|
| SLES | 12 | 12 |
| CAPB | 1.6 | 1.6 |
| Caprylic Acid | 0.1 | 0.2 |
| Coconut oil | 0.25 | 0.25 |
| Water | To 100 | To 100 |
| pH | 4.5 | 4.5 |

Compositions D0 and F0 were non-transparent, indicating that a microemulsion structure had not formed.

Compositions 1 in accordance with the invention, D0 and F0 were stored for 48 hours in an oven at 50 °C, during which D0 and F0 suffered phase separation with the formation of cream on top. The composition in accordance with the invention remained stable.

1. A silicone free aqueous cleansing composition, comprising:
i) from 3 to 15 wt % of a sulphated anionic surfactant;
ii) from 0.5 to 5 wt % of a zwitterionic surfactant;
iii) from 0.1 to 1 wt % a triglyceride oil comprising at least 30% of fatty chains having a carbon-carbon chain length of 12;
iv) from 0.01 to 0.1 wt % caprylic acid; and
v) from 0.1 to 2 wt % of a cationic polysaccharide deposition polymer;
wherein the weight ratio of triglyceride oil to caprylic acid is 5:1 to 15:1;
wherein the composition is in the form of a microemulsion; and
wherein the composition is free from silicone.

2. The composition as claimed in claim 1, wherein the sulphated anionic surfactant is selected from sodium laureth ether sulphate, sodium pareth ether sulphate and mixtures thereof.

3. The composition as claimed in claim 2, wherein the sulphated anionic surfactant has an average degree of ethoxylation of from 1EO to 3EO.

4. The composition as claimed in claim 1, wherein the zwitterionic surfactant is selected from a betaine, cocamide monoethanolamide and mixtures thereof.

5. The composition as claimed in claim 1, which has a weight ratio of the anionic surfactant (i) to the zwitterionic surfactant (ii) of from 3:1 to 10:1.

6. The composition as claimed in claim 1, wherein the triglyceride oil comprises from 30 to 100 wt % of fatty chains having a carbon-carbon chain length of 12.

7. The composition as claimed in claim 6, wherein the triglyceride oil is selected from coconut oil, palm kernal oil, algal oil and mixtures thereof.

8. The composition as claimed in claim 1, wherein the cationic polysaccharide deposition polymer is selected from cationic dextrans, cationic celluloses and cationic polygalactomannans.

9. The composition as claimed in claim 8, wherein the cationic polysaccharide deposition polymer is selected from dextran polymer, cationic cellulose derivative and cationic guar.

10. The composition as claimed in claim 1, wherein the ratio of triglyceride oil to caprylic acid is from 6:1 to 12:1.

11. The composition as claimed in claim 1, which is transparent.

12. A method of treating hair, comprising applying a composition as claimed in claim 1 to the hair.

13. A method of conditioning hair, comprising applying a composition as claimed in claim 1 to the hair.

14. A method to reduce frizz of hair, comprising applying a composition as claimed in claim 1 to the hair, wherein the composition reduces frizz as compared to a composition that does not comprise caprylic acid and triglyceride oil.
